(19) United States
(12) Patent Application Publication
(10) Pub. No.: US 2022/0016093 A1
(43) Pub. Date: Jan. 20, 2022

(54) Compositions for Aiding Liver Function

(71) Applicant: Ajay Suman, Richmond, TX (US)

(72) Inventor: Ajay Suman, Richmond, TX (US)

(21) Appl. No.: 17/299,562

(22) PCT Filed: Dec. 6, 2019
(86) PCT No.: PCT/US19/64947

§ 371 Date: Jun. 3, 2021

Related U.S. Application Data
(60) Provisional application No. 62/776,543, filed on Dec. 7, 2018.

(51) Int. Cl. A61K 31/4375; A61K 31/198; A61K 31/714; A61K 36/28; A61K 31/522

(52) U.S. Cl. CPC A61K 31/4375; A61K 31/198; A61K 31/522; A61K 36/28; A61K 31/714

(57) ABSTRACT

The disclosure relates to compositions that promote liver health. The disclosure also relates to compositions that prevent, mitigate, relieve or reduce the symptoms of hangover.

### BACKGROUND

Amongst its many functions, the liver has a central role in metabolizing, processing or inactivating substances ingested by an individual. For example, the liver metabolizes much of the alcohol and other substances found in alcoholic products. However, the ability of the liver to process these substances may become compromised in a number of situations. For example, an individual's overconsumption of alcoholic products may result in a “hangover” where an individual experiences one or more symptoms including, but not limited to, dehydration, nausea, fatigue, headaches, dizziness, electrolyte imbalance, joint pain and confusion.

The exact mechanism(s) for the development of hangover symptoms is unknown. The symptoms may be due to the accumulation of toxic substances, such as acetaldehyde, because the liver is unable to process completely a large amount of the alcoholic product. Acetaldehyde is toxic and carcinogenic.

Reactive oxygen species (ROS) may also accumulate, possibly due to stress on the liver or other tissues. Most chemical reactions in cells require diatomic oxygen but one or more of the resulting reaction products may be a ROS such as hydrogen peroxide, or free radicals such the hydroxyl ion or the superoxide ion. These ROS may then react with other molecules and/or cellular structures, thereby affecting cellular function. There may be a series of reactions (i.e. a chain reaction) that result from the production of ROS and, consequently, the effects on function may be significant and widespread. In healthy people, cells generally have the capacity to absorb or inactivate ROS such that the undesirable effects are absent or mitigated. Inactivation of ROS may occur by reaction with endogenously synthesized anti-oxidant molecules or complexes. These anti-oxidants may react with ROS and terminate the undesirable reactions.

However, illness or exposure to exogenous or ingested substances may upset the balance between reactive oxygen species and their inactivation, leading to ROS accumulation and resulting cellular damage. The exogenous substances could be ROS themselves or induce the formation of ROS and resulting chain reactions. The result may be oxidative stress where cellular damage due to ROS reactions increases and the health of the individual may be compromised. For example, excessive smoking of tobacco or intake of alcohol may result in oxidative stress. The aging process may result, at least in part, through accumulation of ROS and oxidative stress. As one role of the liver is to inactivate toxic substances, oxidative stress may be particularly impactful on this organ.

The overconsumption of alcohol may also cause immune system changes, hormonal changes and changes in glucose metabolism. The type and severity of particular symptoms may be related to an individual's age, physiological state or the type of alcoholic product ingested. For example, components of alcoholic products other than alcohol may have significant effects on hangover symptoms.

There are numerous products that claim to prevent or reduce the effects of the overconsumption of alcoholic products, but the ness of these products is questionable. There remains a need for an easily consumed product that can prevent, mitigate, relieve or reduce the symptoms of the over-consumption of alcoholic products.

### SUMMARY

The disclosure relates to compositions that prevent, mitigate, relieve, or reduce the symptoms of the over-consumption of alcohol, such as hangover symptoms. The compositions may include N-Acetyl-Cysteine, glutathione, vitamin B9, and vitamin B12. The disclosure also relates to relieving the effects of stress on the liver, such as occurs when toxic products accumulate. The disclosure also relates to methods of using the compositions.

### DESCRIPTION

The present disclosure relates generally to compositions and methods of use that aid in liver health or liver function. Ingestion of compositions of the disclosure may improve liver function or prevent damage to the liver. The compositions may also be useful in improving the function of other tissues or organs.

In preferred examples, the disclosure relates to compositions that prevent, mitigate, relieve or reduce oxidative stress in the liver, however that stress is induced. In preferred examples, compositions of the disclosure may prevent, mitigate, reduce or relieve oxidative stress in the liver, or other organs or tissues. The compositions may prevent, mitigate, relieve or reduce the risk of cellular damage due to ROS reactions, where such reactions may compromise the health of the individual. Compositions of the disclosure may prevent, mitigate, relieve or reduce the undesirable effects of ingested substances on the liver. In preferred examples, compositions of the disclosure, may prevent, mitigate, relieve or reduce the undesirable effects of ingested substances on the liver due to the effects of oxidative stress. In preferred examples, compositions provide at least one component that acts as an anti-oxidant, where the anti-oxidant prevents, mitigates, relieves or reduces the effects of oxidative stress.

In some examples, the present disclosure relates to compositions used to prevent, relieve, mitigate or reduce the effects of the over-consumption of alcoholic products, including the symptoms of hangovers. For example, the composition may prevent, mitigate, relieve or reduce one or more symptoms of hangover including but not limited to dehydration, fatigue, headaches, nausea, dizziness, electrolyte imbalance, joint pain, confusion, vitamin depletion, or nutrient depletion.

Compositions of the disclosure may also prevent the accumulation of ROS, harmful toxins or carcinogens, where the ROS, toxins or carcinogens accumulate through some other cause than over-consumption of alcohol. For example, compositions of the disclosure may be beneficial in relieving the symptoms of the over-consumption of paracetamol.

The disclosure also relates to compositions that provide anti-aging benefits. For example, the compositions may provide anti-oxidants resulting in anti-aging benefits.

The disclosure also relates to the methods of use of the disclosed compositions in various situations. For example, the disclosure describes methods of use for preventing, mitigating relieving or reducing one or more of the symptoms of hangover.

According to the disclosure, a single dose of a composition is generally sufficient to prevent, mitigate, relieve or reduce the effects of ingested substances on the liver. In preferred examples, a single dose may prevent, mitigate, relieve or reduce one or more of the symptoms of hangover. In some examples, a portion or a fraction of single dose may be ingested and still be effective. For example, an individual may find a portion or fraction of a dose to be effective in mitigating one or more symptoms of hangover.

Compositions of the disclosure may take several forms. For example, a single dose may be presented in a single pill, or a single sachet containing the composition in powder form. These forms may be taken or ingested in solid form or may be added to a beverage for consumption. In other examples, a single dose of a composition may be in the form of a liquid such as a “shot” where a consumer is able to consume a single dose of the product quickly. For example, a single dose of a “shot” may be contained in from about one (1) ounce to about five (5) ounces (from about 30 ml to 150 ml). In one particularly preferred example, the product is included in a “shot” having a volume of about two (2) ounces (from about 57 to about 63 ml). In further preferred examples, a composition may have a liquid volume from about 10 ml to about 500 ml. In another preferred example, a composition may have a volume of about 500 ml.

According to the disclosure, compositions include at least three of N-Acetyl-L-Cysteine, glutathione, vitamin B9 or vitamin B12. In preferred examples, compositions include glutathione and at least two of N-Acetyl-L-Cysteine, vitamin B9 or vitamin B12.

In particularly preferred examples, compositions include N-Acetyl-L-Cysteine, glutathione, vitamin B9 and vitamin B12. In preferred examples, the ingredients of the composition create a synergistic effect, enhancing the effects of the individual ingredients.

According to the disclosure, preferred examples of a composition have from about 400 mg of N-Acetyl-L-Cysteine to about 2000 mg of N-Acetyl-L-Cysteine, or from about 800 mg of N-Acetyl-L-Cysteine to about 2000 mg of N-Acetyl-L-Cysteine per dose. In further preferred examples, compositions have from about 800 mg to about 1800 mg of N-Acetyl-L-Cysteine per dose. In particularly preferred examples, compositions have from about 1000 mg to about 1500 mg of N-Acetyl-L-Cysteine per dose.

According to the disclosure, preferred examples of a composition have from about 50 mg of to about 500 mg of glutathione, or from about 80 mg to about 250 mg of glutathione per dose. In particularly preferred examples, compositions have from about 100 mg to about 200 mg of glutathione per dose.

According to the disclosure, preferred examples of a composition have from about 50 mcg of to about 1000 mcg of vitamin B9, or from about 80 mcg to about 750 mcg of vitamin B9 per dose. In particularly preferred examples, compositions have from about 300 mg to about 600 mg of vitamin B9 per dose. In particularly preferred examples, compositions have from about 450 mcg to about 550 mcg of vitamin B9 per dose.

According to the disclosure, preferred examples of a composition have from about 50 mcg of to about 1000 mcg of vitamin B12, or from about 80 mcg to about 750 mcg of vitamin B12 per dose. In particularly preferred examples, compositions have from about 300 mcg to about 600 mcg of vitamin B12 per dose. In particularly preferred examples, compositions have from about 450 mcg to about 550 mcg of vitamin B12 per dose.

According to the disclosure, preferred examples of a single dose of a composition may have from about 1000 mg to about 1300 mg of N-Acetyl-L-Cysteine, from about 100 mg to about 200 mg of glutathione, from about 450 mcg to about 550 mcg of vitamin B12 or from about 450 mcg to about 550 mcg of vitamin B9.

As shown in Table 1, in a particularly preferred example, a single dose of compositions has about 1100 mg of N-Acetyl-L-Cysteine, about 150 mg of glutathione, about 500 mcg of vitamin B12 and about 500 mcg of vitamin B9.

TABLE 1

| Ingredient | Amount | |
|---|---|---|
| N-Acetyl-L-Cysteine | 1100 | mg |
| Glutathione | 150 | mg |
| Vitamin B9 | 500 | mcg |
| Vitamin B12 | 500 | mcg |

For example, according to Table 1, a shot having of a volume of 60m1 would have a concentration of N-Acetyl-L-Cysteine of about 18.3 mg/ml, glutathione at a concentration of about 2.5 mg/ml, vitamin B9 at about 8.3 mcg/ml, and vitamin B12 at about 8.3 mcg/ml.

Compositions of the disclosure may include precursors, structurally-similar molecules, analogs or metabolites of molecules identified in the disclosure where the precursors, structurally-similar compounds, analogs or metabolites are generally functionally similar to the identified molecules. For example, the disclosure encompasses anti-oxidants that are precursors, structurally-similar molecules, analogs or metabolites of the identified molecules described herein.

N-Acetyl-L-Cysteine is an anti-oxidant that may inactivate ROS. For example, N-Acetyl-L-Cysteine is used for the treatment of over-consumption of paracetamol. Glutathione is also an anti-oxidant and is essential to energy utilization, immune system performance and disease prevention. Glutathione is found throughout the body, including the liver.

Vitamins B9 and B12 are important cofactors in a number of enzymatic reactions. For example, vitamin B9 is a cofactor for enzymatic reactions involved in nucleic acid synthesis and synthesis of certain amino acids. Vitamin B12 is involved in energy production and the synthesis of certain amino acids. In preferred examples, vitamins of the composition may replace or supplement nutrients depleted as a consequence of oxidation or other processes, such as might occur during the over-consumption of alcoholic products.

Compositions of the disclosure may also include other components. For example, the composition may include other vitamins, including, for example, other B vitamins, such as choline or vitamin B6. In some examples, vitamin A, vitamin C or vitamin D may be included. In a single dose, vitamin A may be present from about 200 mcg to about 2000 mcg. Vitamin C may be present from about 40 to about 1000 mg per dose. Vitamin D may be present from about 5 mg to about 75 mg.

In some examples, compositions may include components that provide other characteristics. In some examples, compositions of the disclosure may also include components that aid in sleep, particularly after the over-consumption of alcohol. For example, compositions may include melatonin. Melatonin may be present from about 0.2 mg to about 5 mg per dose.

In other examples, compositions may include a methylated xanthine. In these examples, compositions may provide benefits ascribed to methylated xanthines, such as alertness. In preferred examples, the methylated xanthine is caffeine. The methylated xanthine may be present in from about 5 mg to about 250 mg or from about 40 to about 80 mg or from about from about 40 mg to about 250 mg.

In some examples, compositions according to the disclosure include cannabidiol (CBD). CBD may have multiple effects, including sedative effects. Compositions may have from about 5 mg to about 50 mg of CBD per dose. For example, if the composition is delivered in the form of a shot, CBD may have a concentration from about 0.08 mg/ml to about 0.8 mg/ml. In preferred examples, compositions may have from about 10 mg to 30 mg per dose of CBD, or from about 10 mg to about 15 mg per dose of CBD. In other examples, compositions may have from about 16 mg to about 30 mg per dose.

In some examples, compositions may include dihydromyricetin (DHM). Compositions may have from about 50 mg to about 1000 mg of DHM per dose. For example, if the composition is delivered in the form of an energy shot (approximately 60 ml), CBD may have a concentration from about 1.0 mg/ml to about 17 mg/ml. In preferred examples, compositions may have from about 100 mg to 600 mg per dose, or from about 100 mg to about 300 mg per dose. In other examples, compositions may have from about 310 mg to about 500 mg per dose.

In some examples, compositions may include milk thistle. Compositions may have from about 100 mg to about 1000 mg of milkthistle per dose. For example, if the composition is delivered in the form of a shot (approximately 60 ml), CBD may have a concentration from about 1.7 mg/ml to about 17 mg/ml. In preferred examples, compositions may have from about 200 mg to 600 mg per dose, or from about 600 mg to about 1000 mg per dose. In other examples, compositions may have from about 300 mg to about 600 mg per dose.

Compositions according to the disclosure may be flavored. For example, compositions may be flavored using artificial flavorants. Compositions may also include sweeteners. The composition may include natural sweeteners such as sucrose. Compositions may alternatively have artificial sweeteners such as sucralose. The compositions may include preservatives. For example, compositions may include one or more of sodium benzoate, potassium sorbate, or EDTA.

According to the disclosure, compositions may take before, during or after the consumption of alcohol. For example, compositions be taken in anticipation of the over-consumption of alcoholic products. Compositions may also be taken simultaneously during the consumption of alcoholic products. For example, one or more single doses may be taken during the consumption of two to ten alcoholic products. At least one single dose may be taken during the consumption of three alcoholic products, during the consumption of four alcoholic products, during the consumption of five alcoholic products, or during the consumption of six alcoholic products during or after the consumption of seven, alcoholic products, or during the consumption of eight alcoholic products. In preferred examples, at least one single dose is taken after every six alcoholic products consumed. In some examples, one to six doses of a single dose may be taken in a 24-hour period. In preferred examples, one to four doses may be taken in a single 24-period.

Compositions may also be taken after the consumption of alcohol. For example, at least one single dose may be taken before going to bed. The consumption of one or more single doses may aid sleep quality in individuals that have over-consumed alcoholic products, for example, by reducing the symptoms of hangover.

This description provides illustrative examples intended to provide an overview or framework for a person of ordinary kill in the art to understand the claims. It is to be understood that the terminology used herein, is intended to be in the nature of words of description rather than of limitation. Many modifications and variations would be apparent to one of ordinary skill in the art in light of the teachings of the disclosure and these modifications and variations fall within the scope of the claims.

What is claimed:

1. A composition for aiding liver function, comprising:
N-Acetyl-L-Cysteine;
Glutathione; and
at least one vitamin.

2. The composition of claim 1 wherein said vitamin is selected from the group consisting of vitamin B9, vitamin B12 and combinations thereof.

3. The composition of claim 1

4. The composition of claim 1 further comprising cannabidiol.

5. The composition of claim 1 further comprising dihydromyricetin.

6. The composition of claim 1 further comprising milk thistle.

7. The composition of claim 1 comprising from about 1000 mg to about 1800 mg of N-Acetyl-L-Cysteine, from about 100 mg to about 500 mg of glutathione, from about 250 mcg to about 1000 mcg of vitamin B12 or from about 250 mcg to about 1000 mcg of vitamin B9.

8. The composition of claim 1 comprising about 1100 mg of N-Acetyl-L-Cysteine, about 150 mg of glutathione, about 500 mcg of vitamin B12 and about 500 mcg of vitamin B9.

9. The composition of claim 1 wherein the form of said composition is selected from the group consisting of liquids, pills, powders and combinations thereof.

10. The composition of claim 1 wherein said composition is a shot.

11. The composition of claim 1 wherein said composition has a volume from about 10 ml to about 500 ml.

12. The composition of claim 1 wherein said composition is a pill.

13. The composition of claim 1 further comprising caffeine.

14. The composition of claim 1 further comprising melatonin.

15. A composition for mitigating the effects of hangover, comprising:
N-Acetyl-L-Cysteine;
Glutathione;
Vitamin B9;
Vitamin B12;
cannabidiol;
dihydromyricetin;
and
milkthistle.

16. A method of preventing or mitigating the symptoms of hangover, comprising:
a. ingesting a single dose of a composition, said composition comprising:
N-Acetyl-L-Cysteine;
Glutathione;
Vitamin B9;
Vitamin B12;
cannabidiol;
milkthistle; and
dihydromyricetin;
b. monitoring for symptoms of hangover; and
c. ingesting at least one additional dose of said composition if hangover symptoms persist.

17. The method of claim 16 wherein said composition is selected from the group consisting of pills, powders, liquids, and combinations thereof

18. The method of claim 16 wherein said composition is taken before sleep.

19. The method of claim 18 wherein said composition further comprising melatonin.

20. The method of claim 16 wherein said composition is in the form of a shot.
